(19) United States
(12) Patent Application Publication
(10) Pub. No.: US 2025/0248427 A1
(43) Pub. Date: Aug. 7, 2025

(54) PROCESSING METHOD FOR IMPROVING GLUCOSE ABSORPTION CAPACITY OF SEA RICE PUFFING POWDER AND USE OF SEA RICE PUFFING POWDER

(71) Applicant: WUYI UNIVERSITY, Jiangmen (CN)

(72) Inventors: Lei LIU, Jiangmen (CN); Tiange CHEN, Jiangmen (CN); Yongxian LI, Jiangmen (CN); Taowen CHEN, Jiangmen (CN); Jucai XU, Jiangmen (CN); Min XU, Jiangmen (CN); Min WANG, Jiangmen (CN)

(21) Appl. No.: 18/694,476

(22) PCT Filed: Feb. 7, 2024
(86) PCT No.: PCT/CN2024/076600

§ 371 Date: Mar. 22, 2024

(30) Foreign Application Priority Data
Jan. 19, 2024 (CN) 2024100816992

(51) Int. Cl. A23L 7/104; A23L 7/191

(52) U.S. Cl. CPC A23L 7/107; A23L 7/191

(57) ABSTRACT

A processing method for improving a glucose absorption capacity of sea rice puffing powder and use of the sea rice puffing powder are disclosed. The processing method includes the following steps: mixing high-temperature resistant α-amylase with sea rice bran to obtain a mixture; and adding water to the mixture to adjust a content of water, and then performing steam explosion processing on the mixture after being adjusted the content of water. The processing method provided by the present disclosure can solve the problems of dense dietary fiber structure and poor glucose absorption capacity of sea rice, and provides theoretical basis and technical guidance for developing sea rice into special nutritious food for people with hyperglycemia or diabetes.

### CROSS-REFERENCE TO RELATED APPLICATIONS

The present application is a national phase entry under 35 USC § 371 of International Application PCT/CN2024/076600 filed Feb. 7, 2024, which claims the benefit of and priority to Chinese Patent Application No. 202410081699.2, filed Jan. 19, 2024, the entire disclosures of which are incorporated herein by reference.

### TECHNICAL FIELD

The present disclosure belongs to the technical field of food processing, and specifically relates to a processing method for improving a glucose absorption capacity of sea rice puffing powder and use of the sea rice puffing powder.

### BACKGROUND

Sea rice, also known as saline-alkali tolerant rice, which was discovered and cultivated by researcher Chen Risheng in Suixi County, Zhanjiang City Guangdong Province, China in 1986, is the preferred food crop for the development and utilization of coastal tidal flats and saline-alkali lands, and is an effective way to solve food security problems. Sea rice is rich in nutrients and bioactive substances such as starch, dietary fiber, protein and vitamins, but is high in production cost and not obvious in economic benefit. Currently, there are fewer deep-processing products on the market that use sea rice as raw material. It is of great significance to study the deep-processing technology of sea rice and improve its added value in promoting the development of sea rice industry.

Dietary fiber, as the main structural polysaccharide of plant cell walls, has certain hypoglycemic functions, and plays a role mainly by inhibiting α-amylase and adsorbing glucose. Cereal contains dietary fiber mainly concentrated in the bran layer, which is mainly insoluble dietary fiber, has low content of soluble dietary fiber, and has poor adsorption performance for glucose, limiting its application in the field of food. Sea rice is rich in dietary fiber, and thus there is an urgent need to develop a processing technology to improve the glucose adsorption performance of sea rice dietary fiber.

### SUMMARY

An objective of the present disclosure is to provide a processing method for improving a glucose absorption capacity of sea rice puffing powder, thereby solving the problems of dense dietary fiber structure and poor glucose absorption capacity of sea rice.

In order to solve the above problems, the following technical solution is used in the present disclosure: mixing high-temperature resistant α-amylase with sea rice bran to obtain a mixture; and adding water to the mixture to adjust a content of water, and then performing steam explosion processing on the mixture after being adjusted the content of water.

The specific steps are as follows:

S1. Obtaining sea rice bran: adding shelled sea rice to a rice mill for milling, and collecting the rice bran rich in dietary fiber for further processing. A bran layer of sea rice is rich in dietary fiber, which is higher than that of ordinary brown rice, generally more than 60%. Dietary fiber can absorb glucose and has a certain function of lowering blood glucose, so sea rice bran is suitable for an ingredient processed into special food for diabetics. However, sea rice is rich in starch in endosperm and is not suitable for the preparation of the sea rice puffing powder of the present disclosure, but can be used to make rice.

S2. Spraying amylase and adjusting a content of water: spraying high-temperature resistant α-amylase into the sea rice bran prepared in step S1, adjusting the content of water of the rice bran, and stirring to mix the high-temperature resistant α-amylase and water evenly with the rice bran for further processing. Sea rice bran is rich in dietary fiber, with more than 90% of insoluble dietary fiber, and has low content of soluble dietary fiber, which results in its low glucose adsorption capacity and rough taste, so it is necessary to improve its glucose adsorption capacity through processing and modification. The dietary fiber of sea rice bran has a dense structure. In order to loosen the dense structure in the next step of steam explosion processing, high-temperature resistant α-amylase is added to degrade the starch entangled with the dietary fiber and expose the dietary fiber, which facilitates steam explosion processing. In order for the high-temperature resistant α-amylase to better act on starch, an appropriate amount of water needs to be added to make the sea rice bran still remain in a solid powder state rather than a liquid state. Because too much water is added, it is not conducive to steam explosion processing.

S3. Performing steam explosion processing: putting the sea rice bran containing high-temperature resistant α-amylase in step S2 into a preheated steam explosion machine, maintaining at a certain pressure for a period of time after closing a lid of the steam explosion machine, then opening a valve to instantly release a pressure in the steam explosion machine, and collecting sea rice bran puffed by steam explosion; cooling the sea rice bran at room temperature and then pulverizing the same, sieving a resulting pulverized sea rice bran with a 60-mesh sieve, and storing a resulting sieved sea rice bran hermetically to obtain sea rice puffing powder with good glucose absorption capacity. Steam explosion processing is an environment-friendly and efficient food processing technology that can be used to modify dietary fiber. During the processing, only high-temperature and high-pressure water vapor is required, without adding other chemical components. When the valve is opened, the pressure is instantly reduced, and the water is instantly volatilized, causing the raw materials to undergo synergistic effects such as thermal degradation, hydrogen bond destruction, and structural rearrangement at the same time, which can effectively destroy the dense cell wall structure of plants and expose more net structures and hydrophilic groups, making the structure of the insoluble dietary fiber of sea rice bran looser and increasing the content of soluble dietary fiber to improve the effect of absorbing glucose by sea rice bran.

In step S1, sea rice is milled with a rice mill and rice bran layers are collected. A weight of the rice bran layers accounts for 6%-8% of a weight of the sea rice.

In step S2, a mixing ratio of the high-temperature resistant α-amylase to the sea rice bran is 100 U/g-500 U/g. Based on a dry weight of the sea rice bran, an addition amount of the water is 9%-16% of the dry weight of the sea rice bran. The high-temperature resistant α-amylase and water are sprayed to the sea rice bran in a process of stirring the sea rice bran in a mixer to make the two evenly mixed.

In step S3, a filling rate of the sea rice bran in a steam explosion cylinder is less than 30%. When a pressure in the steam explosion cylinder reaches 1.2-1.6 MPa, after maintaining the pressure for 80-100 s, the valve is opened to instantly relieve the pressure, and the sea rice bran puffed by steam explosion is collected.

The present disclosure further provides sea rice puffing powder prepared by the above processing method.

In some embodiments of the present disclosure, a content of a soluble dietary fiber in the sea rice puffing powder is 7%-9%.

In some embodiments of the present disclosure, a glucose absorption capacity of the sea rice puffing powder is 2-8 mmol/g.

In some preferred embodiments of the present disclosure, a glucose absorption capacity of the sea rice puffing powder in a 50 mM glucose solution is 2-3 mmol/g.

In some preferred embodiments of the present disclosure, a glucose absorption capacity of the sea rice puffing powder in a 100 mM glucose solution is 4-6 mmol/g.

In some preferred embodiments of the present disclosure, a glucose absorption capacity of the sea rice puffing powder in a 200 mM glucose solution is 6-8 mmol/g.

The present disclosure further provides use of the above-mentioned sea rice puffing powder in any one of (1)-(3):
(1) preparing food;
(2) preparing medicines for adsorbing glucose; and
(3) preparing cosmetics for adsorbing glucose.

In some embodiments of the present disclosure, the food includes nutritious food for people with hyperglycemia, diabetics and/or obesity.

In some preferred embodiments of the present disclosure, the nutritious food includes at least one of meal replacement powder, meal replacement bar, meal replacement milkshake, meal replacement biscuit and meal replacement porridge.

Compared with the prior art, the present disclosure has the beneficial effects as follows.

According to the present disclosure, sea rice puffing powder with a high glucose absorption capacity is prepared by processing sea rice. The sea rice puffing powder contains a large amount of dietary fiber with a loose structure and can be used to develop nutritious food for special groups such as obese people or diabetics. Moreover, the general enzymatic hydrolysis process needs to work in a liquid system, while the enzyme in the technical solution of the present disclosure plays a role in a solid system, which plays an enzymatic hydrolysis role during the steam explosion process, being beneficial to the modification of the dietary fiber structure. Meanwhile, the present disclosure increases the added value of sea rice and has a guiding role in promoting the healthy development of sea rice processing industry.

### DETAILED DESCRIPTION

The concept of the present disclosure and the resulting technical effects will be clearly and completely described below in conjunction with examples, so as to fully understand the objectives, features and effects of the present disclosure. Obviously, the described examples are only some of, rather than all of the examples of the present disclosure. Other examples obtained by those skilled in the art without creative labor based on the examples of the present disclosure all fall within the scope of protection of the present disclosure.

### Example 1

In this example, a processing method for improving a glucose absorption capacity of sea rice puffing powder was provided, by which the sea rice puffing powder with a good glucose absorption capacity was prepared. The processing method specifically included following steps:
1) Milling of sea rice: milling 100 kg of shelled sea rice with a rice mill, and when the weight of the sea rice decreased by 6%, stopping milling and collecting sea rice bran layers;
2) Spraying of amylase: stirring the 6 kg of sea rice bran collected in step 1) with a mixer, and spraying high-temperature resistant α-amylase and water into the sea rice bran according to an addition amount of 100 U of high-temperature resistant α-amylase per gram of sea rice bran and a spraying amount of water being 9% of the weight of the rice bran under stirring, mixing evenly and then obtaining a mixture; and
3) Steam explosion processing: putting the mixture described in step 2) into a preheated steam explosion cylinder with a filling rate of less than 30%, when the pressure in the steam explosion cylinder reached 1.2 MPa, maintaining the pressure for 80 s, opening a valve to instantly release the pressure, collecting sea rice bran puffed by steam explosion, cooling the sea rice bran at room temperature and then pulverizing the same, sieving the pulverized sea rice bran with a 60-mesh sieve, storing the sieved sea rice bran hermetically, and thus obtaining the sea rice puffing powder with a good glucose absorption capacity.

### Example 2

In this example, a processing method for improving a glucose absorption capacity of puffing sea rice powder was provided, by which the puffing sea rice powder with a good glucose absorption capacity was prepared. The processing method specifically included following steps:
1) Milling of sea rice: milling 200 kg of shelled sea rice with a rice mill, and when the weight of the sea rice decreased by 8%, stopping milling and collecting rice bran layers;
2) Spraying of amylase: stirring the 16 kg of sea rice bran collected in step 1) with a mixer, and spraying high-temperature resistant α-amylase and water into the sea rice bran according to an addition amount 500 U of high-temperature resistant α-amylase per gram of sea rice bran and a spraying amount of water being 16% of the weight of the rice bran under stirring, mixing evenly and then obtaining a mixture; and
3) Steam explosion processing: putting the mixture described in step 2) into a preheated steam explosion cylinder with a filling rate of less than 30%, when the pressure in the steam explosion cylinder reached 1.6 MPa, maintaining the pressure for 100 s, opening a valve to instantly release the pressure, collecting the sea rice bran puffed by steam explosion, cooling the sea rice bran at room temperature and then pulverizing the same, sieving the pulverized sea rice bran with a 60-mesh sieve, storing the sieved sea rice bran hermetically, and thus obtaining the sea rice puffing powder with a good glucose absorption capacity.

### Comparative Example 1

In this comparative example, a processing method for sea rice puffing powder was provided by which the sea rice puffing powder was prepared. The difference between the processing method and that in Example 1 was only that: the step of adding high-temperature resistant α-amylase was not conducted in Comparative example 1.

### Comparative Example 2

In this comparative example, a processing method for sea rice puffing powder was provided by which the sea rice puffing powder was prepared. The difference between the processing method and that in Example 1 was only that: an addition amount of water in step 2) was 3% of the weight of rice bran in Comparative example 2.

### Comparative Example 3

In this comparative example, a processing method for sea rice puffing powder was provided by which the sea rice puffing powder was prepared. The difference between the processing method and that in Example 1 was only that: the steam explosion pressure in step 3) was 0.8 MPa and maintained for 80 s in Comparative example 3.

### Comparative Example 4

In this comparative example, a processing method for sea rice puffing powder was provided by which the sea rice puffing powder was prepared. The difference between the processing method and that in Example 1 was only that: the steam explosion pressure in step 3) was only maintained for 20 s in Comparative example 4.

### Test Example

In this test example, the performances of the sea rice puffing powder prepared in Examples 1-2 and Comparative examples 1-4 were tested. The performances included the content of soluble dietary fiber in the sea rice puffing powder and the glucose absorption capacity of the sea rice puffing powder in glucose solutions with different concentrations. The specific test steps were as follows:

### 1. Testing of the Content of Soluble Dietary Fiber:

The contents of soluble dietary fibers in the sea rice puffing powder prepared in Examples 1-2 and Comparative examples 1-4 were tested according to the Association of Official Analytical Chemists (AOAC) official method 991.43 “Total, Soluble and Insoluble Dietary Fiber in Foods”. The results were shown in Table 1.

TABLE 1
Content of soluble dietary fiber in sea rice puffing powder

| | Key process conditions | Content of soluble |
|---|---|---|
| Group | for steam explosion | dietary fiber (%) |
| Example 1 | Added enzyme, 9% water, | 7.62 ± 0.32d |
| | 1.2 MPa, 80 s | |
| Example 2 | Added enzyme, 9% water, | 7.81 ± 0.41d |
| | 1.6 MPa, 100 s | |
| Comparative | No enzyme, 9% water, | 4.96 ± 0.26b |
| example 1 | 1.2 MPa, 80 s | |
| Comparative | Added enzyme, 3% water, | 4.61 ± 0.22b |
| example 2 | 1.2 MPa, 80 s | |
| Comparative | Added enzyme, 9% water, | 5.28 ± 0.31c |
| example 3 | 0.8 MPa, 80 s | |
| Comparative | Added enzyme, 9% water, | 3.92 ± 0.28a |
| example 4 | 1.2 MPa, 20 s | |

Note:
Different letters in the same column indicated significant differences between values (p < 0.05).

2. Testing of Glucose Absorption Capacity of the Sea Rice Puffing Powder in Glucose Solutions with Different Concentrations:

Three samples of 1 g were weighed from each group, and recorded as m(g). The weighed samples were placed into 10 mL of glucose solutions with concentrations of 50 mM, 100 mM, and 200 mM respectively, recorded the concentration of each glucose solution at this time as c1(mM), and then incubated at 37° C. for 6 h until the absorption of the glucose solution by the sea rice puffing powder reached saturation. The resulting mixtures were centrifuged (1000×g, 20 min), the supernatant was taken, the volumes of the supernatant were recorded as V (mL), and the glucose concentrations of the supernatant were recorded as c2(mM). The glucose concentration was tested by using a D-glucose detection kit, and the glucose absorption capacity was calculated as:

$$Glucose absorption capacity ( mmol / g ) = \frac{( {c}_{1} - {c}_{2} ) \times V}{m} .$$

The results obtained were shown in Table 2.

TABLE 2
Glucose absorption capacity of sea rice puffing powder

| | Glucose absorption capacity (mmol/g) | | |
|---|---|---|---|
| | 50 mM | 100 mM | 200 mM |
| | glucose | glucose | glucose |
| Group | solution | solution | solution |
| Example 1 | 2.61 ± 0.12d | 4.92 ± 0.22d | 6.83 ± 0.32d |
| Example 2 | 2.83 ± 0.12e | 5.14 ± 0.28d | 7.12 ± 0.36e |
| Comparative | 1.56 ± 0.11b | 2.82 ± 0.13b | 4.43 ± 0.21b |
| example 1 | | | |
| Comparative | 1.44 ± 0.13b | 2.66 ± 0.11b | 4.13 ± 0.31b |
| example 2 | | | |
| Comparative | 1.61 ± 0.14c | 3.02 ± 0.16c | 4.83 ± 0.24c |
| example 3 | | | |
| Comparative | 1.28 ± 0.15a | 2.32 ± 0.13a | 3.23 ± 0.16a |
| example 4 | | | |

Note:
Different letters in the same column indicated significant differences between values (p < 0.05).

It can be seen from Table 1 and Table 2 that compared with Example 1, in Comparative example 1, since high-temperature resistant α-amylase was not added before performing the steam explosion processing, the content of soluble dietary fiber in the sea rice puffing powder was significantly reduced, thereby reducing the glucose absorption capacity of the sea rice puffing powder. As can be seen that adding high-temperature resistant α-amylase to assist steam explosion processing will help destroy the structure of insoluble dietary fiber in sea rice, loosen the dense structure of insoluble dietary fiber, and increase the content of soluble dietary fiber, thereby increasing the capacity for sea rice puffing powder to absorb glucose. Compared with Example 1, the water content was reduced in Comparative example 2, the steam explosion pressure was reduced in Comparative example 3, and the time for maintaining the steam explosion pressure was reduced in Comparative example 4, which all resulted in a decrease in the content of soluble dietary fiber in sea rice puffing powder, and a decrease in glucose absorption capacity.

In summary, according to the present disclosure, the content of soluble dietary fiber and glucose absorption capacity of sea rice puffing powder are significantly increased by adding high-temperature resistant α-amylase to assist steam explosion processing. The sea rice puffing powder prepared in the present disclosure can be used for the research, development and production of special food for special groups such as obese patients and hyperglycemic patients, to reduce postprandial blood glucose of patients and improve health conditions of patients. The technology of the present disclosure broadens the ways for deep processing and utilization of sea rice, increases the added value of sea rice, and provides a reference for promoting the development of sea rice processing industry.

The examples of the present disclosure are described in detail above, but the present disclosure is not limited to the above-mentioned examples. Various changes can be made within the scope of knowledge possessed by those of ordinary skill in the art without departing from the purpose of the present disclosure. In addition, the examples of the present disclosure and the features in the examples may be combined with each other without conflict.

1. A processing method for improving a glucose absorption capacity of sea rice puffing powder, comprising:
mixing high-temperature resistant α-amylase with sea rice bran to obtain a mixture; And
adding water to the mixture to adjust a content of water, and then performing steam explosion processing on the mixture after being adjusted the content of water.

2. The processing method according to claim 1, wherein a mixing ratio of the high-temperature resistant α-amylase to the sea rice bran is 100 U/g to 500 U/g.

3. The processing method according to claim 1, wherein an addition amount of the water is 9 w/w % to 16 w/w % of the sea rice bran.

4. The processing method according to claim 1, wherein the steam explosion processing is performed on the mixture in a steam explosion machine.

5. The processing method according to claim 1, wherein a pressure of the steam explosion is 1.2-1.6 MPa.

6. The processing method according to claim 1, wherein the pressure of the steam explosion is maintained for 80 s to 100 s.

7. Sea rice puffing powder prepared by the processing method of claim 1.

8. The sea rice puffing powder according to claim 7, wherein a content of soluble dietary fiber in the sea rice puffing powder is 7% to 9%.

9-10. (canceled)

11. The processing method according to claim 4, wherein a filling rate of the mixture in the steam explosion machine is less than 30%.

12. The sea rice puffing powder according to claim 7, wherein a glucose absorption capacity of the sea rice puffing powder is 2 mmol/g to 8 mmol/g.

13. The sea rice puffing powder according to claim 7, wherein a glucose absorption capacity of the sea rice puffing powder in a 50 mM glucose solution is 2 mmol/g to 3 mmol/g.

14. The sea rice puffing powder according to claim 7, wherein a glucose absorption capacity of the sea rice puffing powder in a 100 mM glucose solution is 4 mmol/g to 6 mmol/g.

15. The sea rice puffing powder according to claim 7, wherein a glucose absorption capacity of the sea rice puffing powder in a 200 mM glucose solution is 6 mmol/g to 8 mmol/g.

16. A food comprising the sea rice puffing powder of claim 7.

17. A medicine for adsorbing glucose comprising the sea rice puffing powder of claim 7.

18. A cosmetic for adsorbing glucose comprising the sea rice puffing powder of claim 7.

19. The food according to claim 16, wherein the food comprises nutritious food for people with hyperglycemia, diabetics and/or obesity.

20. The food according to claim 19, wherein the nutritious food comprises at least one of meal replacement powder, meal replacement bar, meal replacement milkshake, meal replacement biscuit and meal replacement porridge.
